(19) United States
(12) Patent Application Publication
(10) Pub. No.: US 2017/0143284 A1
(43) Pub. Date: May 25, 2017

(54) METHOD TO DETECT A RETAINED SURGICAL OBJECT

(71) Applicant: Carestream Health, Inc., Rochester, NY (US)

(72) Inventors: William J. Sehnert, Fairport, NY (US); Zhimin Huo, Pittsford, NY (US)

(21) Appl. No.: 15/359,808

(22) Filed: Nov. 23, 2016

Related U.S. Application Data
(60) Provisional application No. 62/259,667, filed on Nov. 25, 2015.

(51) Int. Cl. A61B 6/12; A61B 6/00; A61B 90/00; A61B 5/00

(52) U.S. Cl. CPC A61B 6/12; A61B 5/7264; A61B 6/468; A61B 6/463; A61B 6/5258; A61B 90/361; A61B 6/4441; A61B 2090/0805

(57) ABSTRACT

An imaging method, executed at least in part by a computer, tracks the disposition of surgical supplies used in an operation and identifies a radiographic imaging technique for detecting a retained surgical foreign object according to the tracking. One or more radiographic images are acquired in the operating room. The acquired image content is analyzed to identify one or more candidate foreign objects. At least a portion of the acquired image content is displayed with the one or more candidate foreign objects in the acquired image content highlighted.

### CROSS REFERENCE TO RELATED APPLICATIONS

This application claims the benefit of U.S. Provisional application U.S. Ser. No. 62/259,667, provisionally filed on Nov. 25, 2015, entitled “METHOD TO DETECT A RETAINED SURGICAL INSTRUMENT”, in the names of Zhimin Huo et al., incorporated herein in its entirety.

### TECHNICAL FIELD

The disclosure relates generally to the field of medical imaging, and in particular to methods and apparatus for imaging and detection of a retained surgical instrument or other foreign object.

### BACKGROUND

A retained surgical foreign object (RSFO) is an item inadvertently left behind in a patient's body in the course of surgery. This can include a surgical instrument, needle, sponge, or other material that remains in the wound following wound closure. The consequences of retained surgical tools and materials can include the need for repeated surgery, excess monetary cost, loss of hospital credibility, risk of injury or complication, and, in extreme cases, death of the patient.

According to at least one article, thousands of patients a year leave the nation's operating rooms with various surgical items in their bodies. And despite occasional instances of forceps, clamps, and other hardware showing up in post-operative X-rays, prominent hardware items are rarely the problem. A problem more frequently encountered and troublesome for the patient and surgical team is gossypiboma, a term used for a condition in which a sponge, towel, gauze, or other soft item is retained in the wound area following surgery. Reference http://www.usatoday.com/story/news/nation/2013/03/08/surgery-sponges-lost-supplies-patients-fatal-risk/1969603/ (dated Mar. 8, 2013), incorporated herein by reference.

One article indicates that sponges present the biggest problem, accounting for about 70% of lost surgical items. By comparison, needles account for less than 10% of RSFOs; instruments account for about 5%.

There can be serious consequences when this occurs. Many patients carrying surgical sponges suffer for months or years before gossypiboma is diagnosed as the cause of the searing pain, digestive dysfunction, and other typical ills. Often, by the time the error is discovered, infection has set in.

To help prevent occurrence of RSFOs, some hospitals routinely count sponges and gauze pads. Other hospitals use electronic technologies to reduce the risk of sponges being left in patients. For example, some hospitals use sponges equipped with electronic tracking devices, bar codes, and radio-frequency detection systems.

Tracking comes at a price. It is estimated that sponge-tracking systems typically add around $10 to the cost of an operation, which is a small fraction of the average procedure's price. But with hospitals performing many thousands of surgeries a year, there is an investment despite possible savings in liability costs. As hospitals work to constrained budgets, they evaluate how to invest scarce resources in achieving safer care for their patients.

Various detection methods have been tried, but found often unsatisfactory. When doctors suspect a sponge has been lost, for example, they can capture a 2D radiographic projection (X-ray) image. However, this procedure typically does not happen unless a sponge count shows a discrepancy. Even when an image is obtained, however, indications are that a lost sponge can be difficult to spot on the x-ray image. One article indicates that there is a problem with detecting these cases once they occur, noting that there are numerous case reports where patients don't present (symptoms) for months, years, sometimes decades.

In response to a problem with sponge RSFOs, the Mayo Clinic began requiring post-operative X-rays for surgical patients, regardless of routine sponge and instrument counts. If scans show a problem after wound closure, another surgery may be needed to retrieve any items that were spotted. To avoid such additional surgeries, some hospitals have adopted sponge-tracking system where each sponge has a unique bar code that is scanned before and after it goes into a patient.

Wikipedia (see: https://en.wikipedia.org/wiki/Retained_surgical_instruments, incorporated herein by reference) indicates that various techniques have been put into practice to prevent gossypiboma. These include the following:

(i) Radiopaque marking, Before operation, sponges can be soaked through with radio-opaque marker. This allows a sponge to be seen on plain radiographs. When the markers are noticed, it can be assumed that it is revealing a retained sponge. Some believe this method has flaws if the sponges have broken into smaller pieces over time.

(ii) Ultrasonography—Gossypiboma can be recognized with ultrasonography by the presence of brightly echogenic wavy structures in a cystic mass showing posterior acoustic shadowing that changes in parallel with the direction of the ultrasound beam.

(iii) Computerized Tomography (CT)—A surgical sponge on a CT will show air bubbles on soft tissue masses. Though some believe there is a concern with this technique is that gossypibomas are easily confused with abscesses.

One proposed method uses pattern recognition to help detect various types of candidate RSFO in an x-ray obtained immediately following surgery. This type of approach may work effectively for surgical instruments formed of dense, radio-opaque metals. However, pattern recognition is not well suited for detection of sponges and absorbent materials that can be retained in the wound area.

Reference is made to U.S. Pat. No. 9,317,920 (Gluncic) and WO 2011/103590 (Asiyanbola).

A retained surgical instrument is a preventable medical condition, and there is a need for a method to detect retained surgical instruments within a patient, preferably in the surgical area, prior to surgical wound closure.

### SUMMARY

Certain embodiments described herein address the need for improved detection of foreign objects retained in the body of a patient following a surgical procedure. According to an embodiment of the present disclosure, a method is described that would allow detection prior to wound closure.

These aspects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

According to an embodiment of the present disclosure, there is provided an imaging method, executed at least in part by a computer, the method comprising: tracking the disposition of surgical supplies used in an operation; identifying a radiographic imaging technique for detecting a retained surgical foreign object according to the tracking; acquiring one or more radiographic images in the operating room; analyzing the acquired image content to identify one or more candidate foreign objects; displaying at least a portion of the acquired image content, highlighting the one or more candidate foreign objects in the acquired image content.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 shows a collection of sponge and gauze pads typical of those used in surgical procedure.

FIG. 2A is a schematic diagram that shows a conventional imaging apparatus for fluoroscopy or x-ray image acquisition.

FIG. 2B is a schematic diagram that shows a portable imaging system for fluoroscopy or x-ray imaging.

FIG. 2C shows a side view of a portable imaging apparatus that can be used for providing a radiation source for use in an operating room environment.

FIG. 2D shows a schematic diagram of an imaging apparatus for tomosynthesis.

FIG. 2E shows a schematic diagram of a portable imaging apparatus for computed tomography.

FIG. 3 is a logic flow diagram that shows a sequence for retained object detection and reporting according to an embodiment of the present disclosure.

FIG. 4A shows a low-dose tomography image of a patient having a retained surgical material.

FIG. 4B shows a conventional posterior-anterior (PA) image showing the retained object of FIG. 4A, enlarged.

FIGS. 4C and 4D shows results of imaging processing using computer-aided diagnostic (CAD) analysis and display software.

FIG. 5A shows a conventional fluoroscopy image of a patient that exhibits high noise content and low contrast.

FIG. 5B shows the content of FIG. 5A with enhanced processing and rendering, showing effects of spatial frequency decomposition processing.

FIGS. 5C and 5D show exemplary results of rib suppression.

FIG. 6 is a schematic view that shows the use of a transport apparatus with a portable imaging system.

FIGS. 7A and 7B are schematic top views that show changing receiver position using a transport apparatus.

FIG. 8 is a logic flow diagram that shows a sequence for detection of a surgical foreign object according to an embodiment of the present disclosure.

### DETAILED DESCRIPTION OF THE EMBODIMENTS

The following is a detailed description of embodiments of the invention, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used as labels to more clearly distinguish one step, element, or set of elements from another, unless specified otherwise.

As used herein, the term “energizable” relates to a device or set of components that perform an indicated function upon receiving power and, optionally, upon receiving an enabling signal.

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present disclosure, the term “coupled” is intended to indicate a mechanical association, connection, relation, or linking, between two or more components, such that the disposition of one component affects the spatial disposition of a component to which it is coupled. For mechanical coupling, two components need not be in direct contact, but can be linked through one or more intermediary components.

In the context of the present disclosure, the terms “viewer”, “operator”, and “user” are considered to be equivalent and refer to the viewing practitioner or other person who can obtain, view, and views manipulate a radiographic image on a display monitor.

The term “highlighting” for a displayed feature has its conventional meaning as is understood to those skilled in the information and image display arts. In general, highlighting uses some form of localized display enhancement to attract the attention of the viewer. Highlighting a portion of an image, such as an individual surgical instrument, material, feature, or other structure, for example, can be achieved in any of a number of ways, including, but not limited to, annotating, displaying a nearby or overlaying symbol such as an arrow, outlining or tracing, display in a different color or at a markedly different intensity or gray scale value than other image or information content, blinking or animation of a portion of a display, or display at enhanced sharpness or contrast. In the image processing context of the present disclosure, “rendering” is the active process of generating and forming an image for display and generating the pattern of signals needed for displaying it to a user. Image data content that is used for rendering can be transformed from a 2D or 3D model (or models), typically stored as scene content in some type of scene file, into suitable patterns of light energy that are emitted from a display screen. A scene file contains objects in a strictly defined language or data structure, describing aspects of the image content such as geometry, viewpoint, texture, lighting, and shading information as a description of a scene. The data contained in the scene content or scene file is passed to a rendering program to be processed and output or streamed to a display driver or graphics processing unit (GPU) for direct presentation on a display or to a digital image or raster graphics image file. The digital image data file can alternately be available for presentation on a display. In general, the term “rendering” provides a transformation that can be considered as analogous to an “artist's rendering” of a scene; different artists working in different media can generate different renderings of the same scene content.

Continuing development of portable radiography apparatus now makes it possible to acquire radiographic images at the patient bedside, including an operating room environment. Portable systems, including mobile systems, have now become available for tomosynthesis and dual-energy (DE) imaging, methods well known for medical imaging. Such systems and methods for their effective use are diagnostic resources readily available and employed by hospitals, clinics, and other health care facilities. Increased portability of these systems allows their use in the operating room, eliminating the need to remove the patient from the surgical area in order to obtain a tomosynthesis or DE image. There can be particular advantages in obtaining multiple images of a surgical region of interest (ROI), where each image provides information from a different aspect, such as having different energy level from DE imaging or having different angle, such as from limited tomosynthesis or tomography imaging.

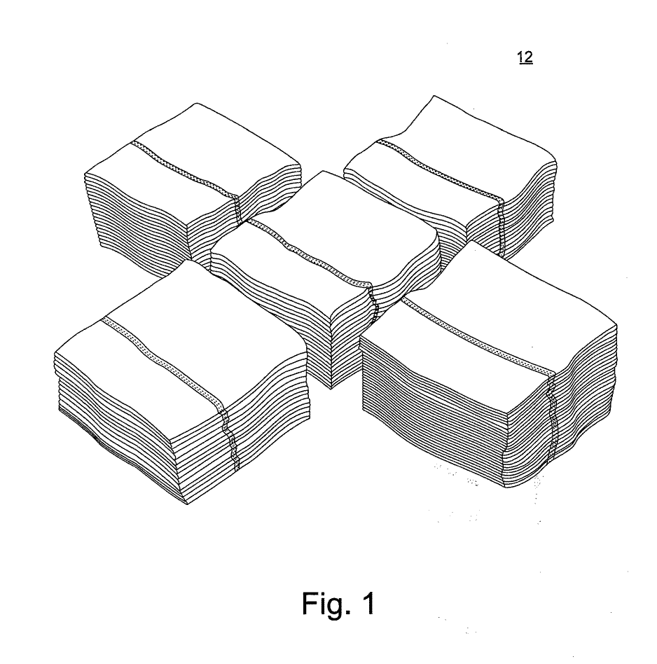

Applicants have recognized that some types of surgical instruments, tools, and supporting materials are composed of or contain metals and other substances that are highly attenuating or absorbent to x-rays and have radio-opaque properties similar to those of dense bone. Among surgical instruments that can be less radio-opaque are sponges, towels, and gauze pads 12, as shown in FIG. 1.

Accordingly, Applicants have recognized that Dual Energy (DE) imaging, which has the ability to separate bone from soft tissue, can be employed in order to improve the detectability of instruments and materials inside the body, suppressing or eliminating bone or soft tissue content from the acquired image for enhanced display of features of particular interest, including candidate RSFOs.

Applicants have further recognized that bone (e.g., rib) structure can be suppressed and/or removed from the radiographic image in order to further improve the detectability of retained instruments and materials in DE images.

Applicants have further recognized that bone (e.g., rib) structure can be suppressed from the radiographic image in order to further improve the detectability of retained instruments and materials in standard chest images.

Applicants have further recognized that tomosynthesis imaging can be employed to improve the detectability of retained instruments inside the body, such as within a surgical region of interest (ROI).

Applicants have also recognized that the combination of Dual Energy and tomosynthesis imaging can be employed to improve the detectability of retained instruments and devices inside the body.

### Radiographic Imaging Apparatus

Various types of radiographic imaging apparatus can be used for acquiring one or more images suitable for candidate RSFO detection. These apparatus include x-ray apparatus, tomosynthesis apparatus, fluoroscopy apparatus, dual-energy x-ray apparatus, and volume imaging systems such as computed tomography (CT) or cone-beam computed tomography (CBCT) apparatus.

Tomosynthesis, also referred to as digital tomosynthesis, is a method for performing high-resolution limited-angle tomography at radiographic dose levels. It has been studied for a variety of clinical applications, including vascular imaging, dental imaging, orthopedic imaging, mammographic imaging, musculoskeletal imaging, and chest imaging. As noted in Wikipedia, tomosynthesis combines digital image capture and processing with simple tube/detector motion as used in conventional computed tomography (CT). However, though there are some similarities to CT, tomosynthesis is a separate technique, performed by dedicated systems. In CT, the source/detector makes at least a complete 180-degree rotation about the subject, obtaining a complete set of data from which volume image content can be reconstructed. Digital tomosynthesis, on the other hand, uses only a limited angular rotation with respect to the subject (e.g., 15-60 degrees) with a reduced number of discrete exposures (e.g., 7-51) than CT. This incomplete set of projections is digitally processed to yield images similar to conventional tomography, but with a more limited depth of field. Because the image processing is digital, a series of slices acquired at different depths and with different thicknesses can be reconstructed from the same acquisition. However, since fewer projections are needed than with CT in order to perform volume reconstruction, radiation exposure and cost are both reduced with tomosynthesis. Reconstruction algorithms for tomosynthesis provide correspondingly lower resolution when compared against conventional CT. Iterative algorithms based upon expectation maximization are most commonly used, but can be computationally intensive. Some manufacturers have produced practical systems using off-the shelf GPUs to perform the reconstruction and image rendering within a few seconds.

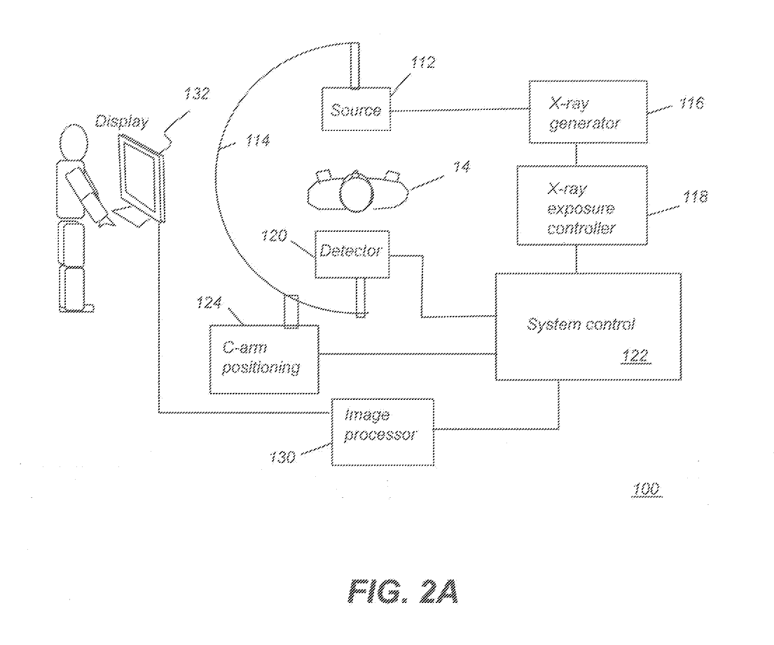

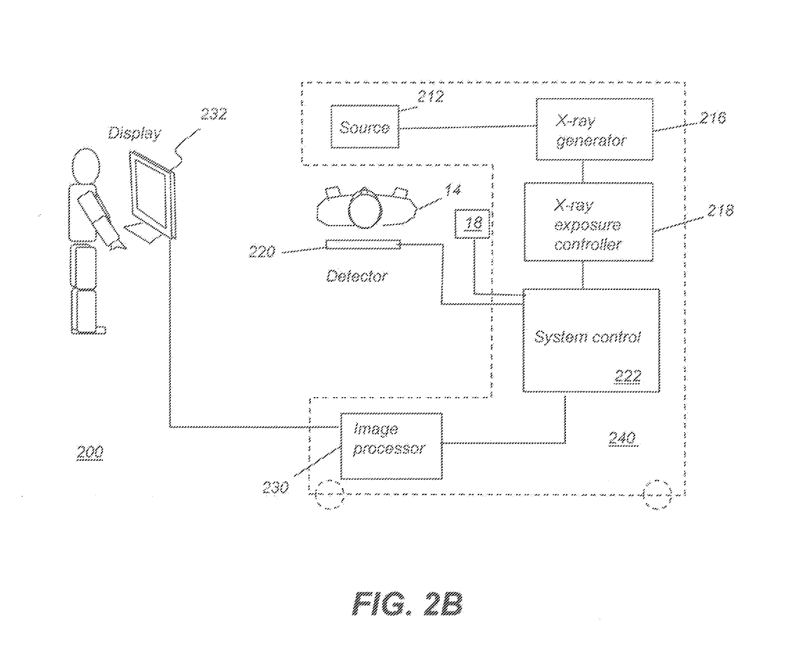

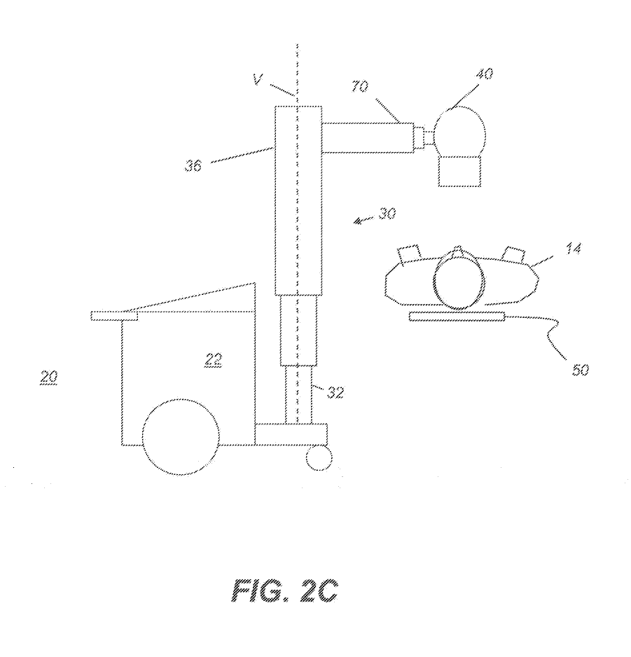

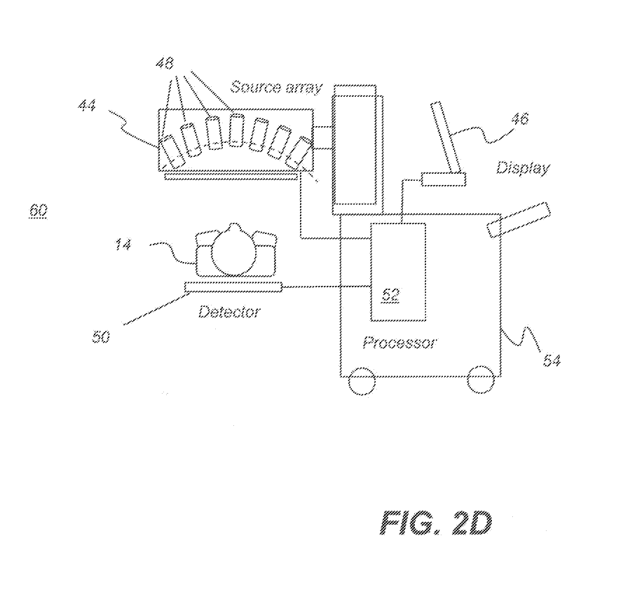

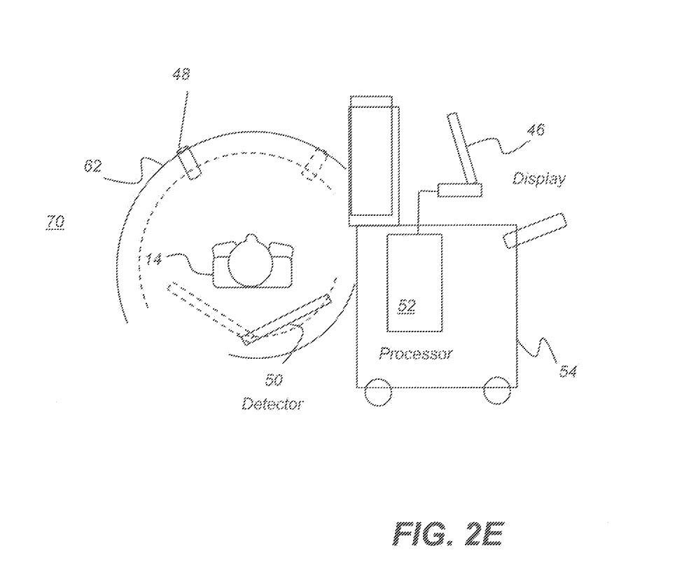

The block diagram of FIG. 2A shows components of a conventional x-ray or fluoroscopy imaging apparatus 100 using mechanically coupled source and detector components. A radiation source 112 and detector 120 are mounted on a C-arm 114 that allows adjustable positioning about a patient 14 or other subject. Source 112 is supplied by an X-ray generator 116, controlled by an exposure controller 118. A system controller 122 coordinates x-ray generation and image acquisition timing, along with positioning of the C-arm 114 by control of a C-arm positioner/transport 124. An image processor 130 obtains and processes the acquired image data and presents the fluoroscopy image sequence on a display 132. In the conventional apparatus 100, detector 120 can include an image intensifier tube or other component that connects to controller 122.

The block diagram of FIG. 2B shows some components of a portable radiography imaging system 200. In this configuration, a detector 220 is a digital radiography DR detector that is mechanically uncoupled from the x-ray radiation source 212. DR detector 220, placed beneath or to the side of the patient 14, communicates through a cabled or wireless connection to a system controller 222. A dashed outline indicates components that can be part of a portable radiographic imaging apparatus 240, such as a portable radiography apparatus provided on a mobile cart that can be wheeled between different locations within a hospital or other medical facility and maneuvered into position by an operator, such as by a member of the surgical team. Apparatus 240 can include an X-ray generator 216, and an X-ray exposure controller 218 under control by system controller 222. An image processor 230, in signal communication with system controller 222, then performs the needed image processing on the acquired image or image sequence and presents the processed image content for the subject on a display 232 that can be part of the portable apparatus 240 or can be a separately provided device that is in signal communication with image processor 230. Display 232 can provide an operator interface with an operating mode for on-site imaging of a surgical region of interest.

Components not shown in the simplified schematic diagrams of FIGS. 2A and 2B can include supporting hardware for transport, power, network connection to storage devices, and other standard components provided with or available to radiographic imaging systems.

Radiography apparatus such as those shown in FIGS. 2A and 2B can acquire, process, and render or display successive images of a patient or other type of subject in rapid sequence. With fluoroscopy, the displayed content has the appearance of video display as the image sequence is rendered to the display. Fluoroscopy imaging is advantaged in being capable of showing motion of and within internal anatomy such as for positioning tubing or other device. Conditioning of the image content in the ongoing fluoroscopy sequence must be performed at high speeds and is typically performed equivalently for all images in the sequence. Image quality is not optimized for fluoroscopy viewing; however, there can be significant utility in using fluoroscopy image capabilities for foreign object detection, as described in more detail subsequently.

In general, there is a proportional relationship between radiation dose levels and image processing. The higher the dose, the more detail available for image processing. Thus, image processing can have increased density of information at higher dose and image processing algorithms and techniques can take advantage of this increased density by using more aggressive parameters, with extended inherent dynamic range and other characteristics, for example.

Portability and mobility are useful attributes of an imaging apparatus for operating-room use. FIG. 2C shows a side view of a portable, mobile imaging apparatus 20 that can be used for providing a radiation source 40 for use in an operating room environment. Imaging apparatus 20 can be an x-ray apparatus or a fluoroscopy apparatus, for example. Apparatus 20 has a portable cart 22 with an expandable column 30 for positioning source 40 suitably for patient 14 imaging. Column 30 has sections 36 and 32 configured in a telescoping arrangement, adjustable along a vertical axis V. A portable digital radiography (DR) detector 50 can be positioned in a suitable position beneath or alongside the, patient 14. The FIG. 2C configuration can also be used for tomosynthesis, acquiring a series of images of a region of interest (ROI) by incremental translational or angular movement of either the radiation source or the detector, or of both.

FIG. 2D shows one alternate type of portable tomosynthesis imaging apparatus 60 on a cart 54 that can be used for generating one or more radiographic images of the surgical ROI and presenting these on a display 46. A source array 44 can have multiple x-ray sources 48 for acquiring images of the ROI at suitable angles. A processor 52 acquires the image content from a portable detector 50 in order to generate one or more images for analysis and RSFO detection. Alternately, source 48 can be a single radiation source that is translated over an arc to capture images of the patient 14 at successive angular increments.

FIG. 2E shows a schematic diagram of a portable imaging apparatus 70 for computed tomography. For the purpose of RSFO detection, a transport apparatus 62 synchronously moves source 48 and detector 50 for acquiring reduced-dose images at two or more different angular positions about patient 14. Imaging apparatus 70 is provided with a cart 54 that can be wheeled to the side of an operating table and configured to provide the limited arcuate travel paths needed for source 48 and detector 50 orbit of patient 14.

Dual-energy (DE) imaging generally involves acquiring one or more paired images at two X-ray energies and processing these images to suppress either the bone or the tissue information. Dual-energy (DE) radiography can be used to eliminate bone information from the surgical ROI in a radiograph, so that an image that displays only tissue content can be displayed. Alternatively, the technique can be used to generate the reverse effect, wherein tissue information is eliminated and an image displaying only bone or dense material content is generated.

Since Applicants have recognized that bone (e.g., rib) structure can be suppressed and/or removed to further improve the detectability of retained foreign surgical objects in DE or standard X-ray images, bone suppression can be performed on the captured image prior to analyzing the captured image to help detect a retained surgical instrument within the surgical ROI. In addition, tissue suppression can also or alternately be performed in order to minimize or remove tissue content that can otherwise obstruct the view of a foreign object or material.

According to an embodiment of the present disclosure, the apparatus used for surgical ROI imaging can be a dedicated system that is specifically designed for this purpose, as described with reference to radiography system 200 in FIG. 2B and optionally combining various additional features shown in schematic form in FIGS. 2A, 2C and 2D, with the option for providing CT imaging as in the system of FIG. 2E. Some desirable features of the apparatus include the following:

(i) Portability. In order to be used effectively in the surgery and post-operative environment, the system should have a high degree of portability, such as being a mobile system that allows appropriate positioning of the radiation source.

(ii) Optional capability for acquiring different types of images. Different types of radiographic imaging have different strengths and advantages that can support RSFO detection. According to an embodiment of the present disclosure, the radiographic imaging apparatus can be used to acquire a single x-ray image, one or more pairs of dual-energy images, or multiple images at different angles, such as using tomosynthesis imaging capability. The system can acquire a full set of 2-D projection images for tomosynthesis or a partial set, having multiple images but not the full tomosynthesis set.

(iii) Display capability, for viewing by the surgical team following image acquisition.

(iv) Display enhancement, indicating areas of abnormality and suspicious regions that should be analyzed by the surgical team. These areas can be highlighted for the viewer using color, increased brightness or density, or other display characteristics.

(v) Adjustable field of view (FOV). The field of view of an imaging system configured for this purpose is variable and can be reduced over that required for conventional radiographic practice, since the surgical area may represent only a small portion of the body. FOV adjustment can be performed using a collimator, for example. The apparatus can automatically control the positioning of radiation source and the detector and sizing of the FOV. This function will effectively reduce the time to set up the system and improve the image quality.

(vi) Optional capability to track surgical instrument and materials use during surgery. This optional capability would allow the system to serve as a tracking system and can provide system logic with useful information that can be used to determine which type of imaging modality to use for detection.

Alternately, the RSFO detection function can be performed using a conventional portable radiographic imaging system, with the system set to a particular mode of operation. Thus, for example, a tomosynthesis system having a typical set of acquisition angles may acquire 60-100 images in conventional imaging operation, with images obtained at 1-degree rotational angle increments. With selection of a surgical ROI imaging mode, as described herein, only a small number of images is acquired, such as one image at every 10- or 12-degree angular position or at incremental positions every few millimeters.

Where multiple imaging modes are available from a single system, a suitable mode or combination of modes can be selected. Imaging modes can be optimized and customized for the detection of particular types of potential retained foreign objects.

Radiographic images for RSFO detection and display can be acquired before or following wound closure. Auto-positioning can be used to position and detect the DR detector.

### Tracking Utilities

The system shown in FIG. 2B can include an optional tracking mechanism 18 that provides additional support for counting and tracking of surgical instruments and materials. Tracking mechanism 18 can include one or more imaging apparatus, such as a camera, a radio-frequency (RF) detection apparatus that tracks RFID tags on individual instruments or disposable materials, a data entry keypad or touch screen that enables manual entry of count totals by an operator and recording of materials or instruments used, or other devices that help to track components used for surgical support. Audio tracking can also be provided by tracking mechanism 18, along with translation capability for extracting data on instruments and supplies usage during surgical procedure. Tracking mechanism 18 can be in signal communication with controller 222 for reporting useful data related to surgical device use and disposition following the surgical procedure.

### Image Processing and Analysis

According to an embodiment of the present disclosure, the captured radiographic image(s) can be analyzed for candidate RSFOs using a computer-aided detection (CAD) algorithm or other knowledge-based expert system. A CAD detection system with a software interface would be configured to show the CAD results (i.e., location of surgical instrument or material within the patient) to a viewing practitioner. With this location information, the instrument or material can be identified and retrieval plans implemented. CAD detection system results can show an approximate location of the instrument or material, thereby assisting the physician with its removal. For example, an implementation would provide an image of the patient, with the foreign objects highlighted on a display for enhanced visibility.

As indicated above, the step of analyzing can include using a computer-aided detection (CAD) algorithm/system or other knowledge-based or expert-based system. CAD and other knowledge-based expert systems are well known. CAD technology can help to pinpoint suspicious areas on medical images by analyzing the shape, groupings, and other characteristics of abnormalities and determining their correlation to previously analyzed disease, characteristics. One example of this type of system is the computer-aided detection (CAD) technology from Kodak/MiraMedica, Inc. This technology solution includes software that automatically highlights suspicious areas on patients' digital medical images or digitized film images, signaling the physician/radiologist to examine these areas.

See for example techniques described in the following patents: U.S. Pat. No. 7,756,317 (Huo), U.S. Pat. No. 8,064,675 (Huo), and U.S. Pat. No. 8,073,229 (Huo), each of which is incorporated herein in its entirety by reference.

Enhancement of RSFOs can be provided for fluoroscopy imaging applications using multi-band frequency decomposition, as described in U.S. Pat. No. 7,848,560 (Wang), incorporated herein by reference in its entirety.

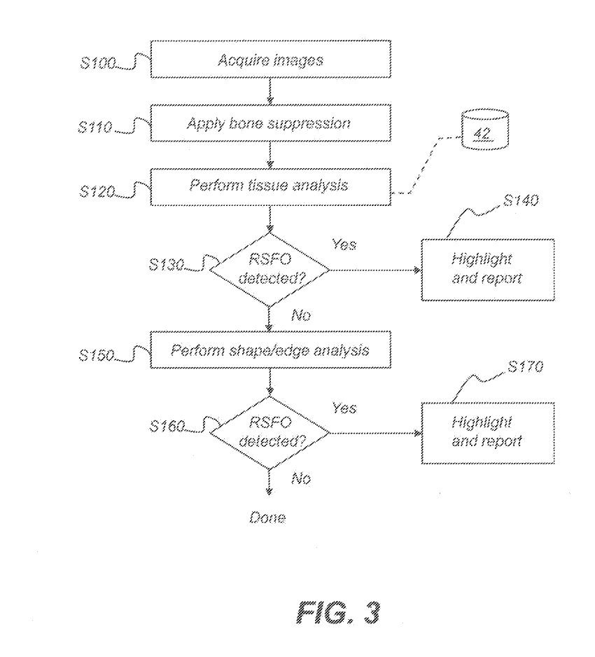

The logic flow diagram of FIG. 3 shows a sequence for RSFO detection and reporting according to an embodiment of the present disclosure. In an acquisition step S100, the system acquires one or more radiographic images of the surgical region of interest (ROI). An optional bone suppression step S110 then applies image processing to suppress bone content, enhancing tissue content for the ROI. A tissue analysis step S120 then analyzes tissue content and compares characteristics of the image tissue with expected tissue characteristics, which can be from stored image content, such as from a database 42. Database 42 can include an atlas that characterizes typical tissue features from a statistical population based on age, sex, weight, and other factors. Alternately, database 42 can include images of the patient taken prior to the surgery. As another alternative, various tissue features can include measurements based on image content and indicative of tissue texture and other characteristics. A decision step S130 then checks the analysis results to determine whether or not an RSFO has been detected or is suspected within the surgical ROI. Where an RSFO is indicated, a highlight and reporting step S140 provides this information for the viewer, in the form of an enhanced display with alternate message content.

Continuing with the FIG. 3 sequence, a subsequent shape/edge analysis step S150 executes. This optional step can detect well-defined edges, highly symmetric or geometric shapes, or other image patterns that can indicate a surgical tool or material in the surgical ROI image. This step can include a library of standard anatomy or an atlas that provides image content for normal tissue or bone features. This step can also use a library of known surgical instruments or materials as a reference for detected image content. A subsequent decision step S160 then determines whether or not an RSFO has been detected according to shape or edge characteristics. Where an RSFO is indicated, a highlight and reporting step S170 provides this information for the viewer, in the form of an enhanced display with alternate message content.

The process flow shown in FIG. 3 can be modified and used for X-ray image, tomosynthesis, dual-energy, fluoroscopy, and CBCT imaging apparatus, with corresponding changes for image acquisition and processing. In image acquisition step S100, for example, a set of two or more images of the same surgical ROI can be obtained. The images within the set can differ from each other by any of a number of aspects that can provide added dimension to the acquired data. Dual-energy (DE) imaging, for example, acquires two images captured from the same angle or perspective, but at different energy levels, such as applying different settings for kVp or mAs values, for example. The higher energy image provides improved visibility of bone structure; lower energy content provides improved visibility of soft tissue.

Alternately, image acquisition step S100 of FIG. 3 can acquire a set of two or more images of the surgical ROI taken at different angles. Acquisition at different angles provides a measure of depth information that is not otherwise available for a single 2-D radiography image. In order to acquire images at different angles, the method and apparatus of the present disclosure can employ a tomosynthesis system having a reduced number of images when compared to the conventional tomosynthesis sequence, for example. A computed tomography (CT) system can alternately be employed, again providing images at a reduced number of angles.

Low-dose tomosynthesis or tomography images can be advantageous for detection of sponge and gauze materials.

It should be noted that automated analysis tools such as the CAD utilities described herein are used to provide assistance to the surgical team while viewing post-operation results. The algorithms and processing used for this purpose detect and report any tissue conditions, edge features, or other features that appear to be different from expected characteristics and that appear to indicate a candidate RSFO. Thus the image analysis focuses on detection and analysis of normal anatomic structures including both bone and soft tissue. The image analysis detects and recognizes individual features such as, heart, liver, ribs, lung, kidney, etc., and can indicate any significant, quantifiable difference from normal anatomy structures, in terms of shape and contents. Further, the image analysis can remove or suppress the normal bone and/or tissue structures on the images, similar to what has been shown for rib suppression. The display of suspicious areas can show the original images with indications of suspicious areas, or images with partially-removed or suppressed normal structures, with an intention to show only the potential foreign objects remaining in the images. The surgical team must analyze the displayed data in order to determine whether or not an RSFO has actually been identified and to determine the course of action based on their assessment of displayed results.

### Results Reporting and Display

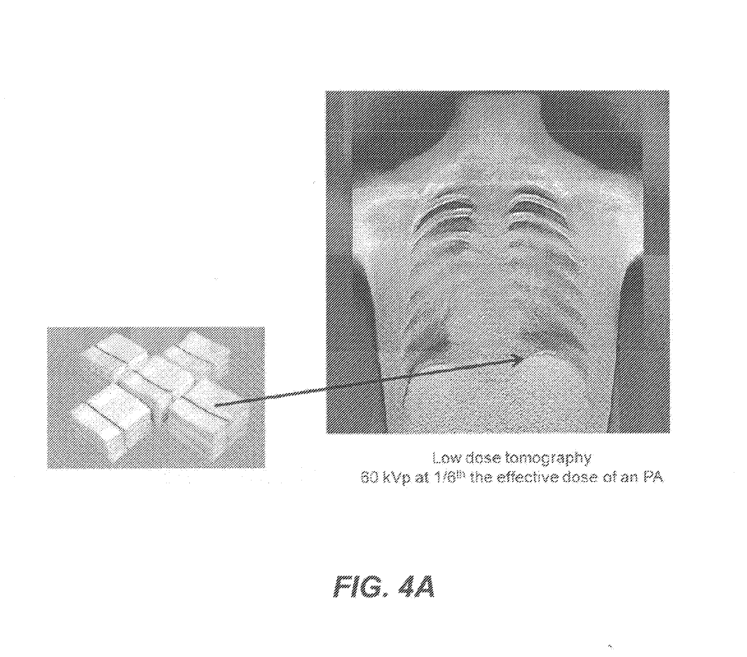

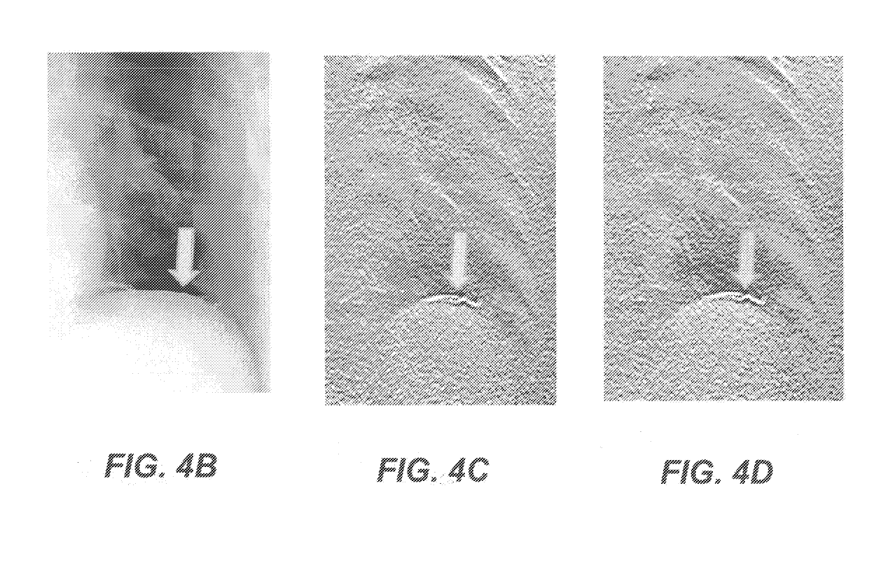

FIGS. 4A-4D shows exemplary images of a sponge/pad within an image of the surgical ROI for a patient using various imaging techniques.

FIG. 4A shows an exemplary low dose tomography image of a sponge/pad within an image of a patient. The low dose tomography image is acquired at 60 kVp, ⅙^{th} the effective dose of a posterior-anterior (PA) image.

The system can automatically zoom to enlarge an area that appears to include a sponge or other RSFO type. Alternately, the system can simply zoom to show the surgical ROI.

FIG. 4B shows a standard PA image of the same content as FIG. 4A, enlarged. As is well known, a PA image is the standard chest radiograph is acquired with the patient standing up, and with the X-ray beam passing through the patient from Posterior to Anterior (PA).

FIGS. 4C and 4D provide a significantly improved view of the sponge that is difficult to discern in FIG. 4B. In FIGS. 4C and 4D, a CAD or other knowledge-based expert system has analyzed the image and provides an indication/indicator (shown in the figures as an arrow) of the location of the retained surgical instrument. The indicator can include an arrow, symbol/marker, bolding, highlighting, coloring, outlining, or the like.

FIG. 4C is an enlarged view of a low dose tomography image corresponding to FIG. 4B, acquired at 60 kVp, ⅙^{th} the PA effective dose.

FIG. 4D is a low dose DE/bone tomography image corresponding to FIG. 4B, acquired at 60 kVp and 120 kVp, ⅓^{rd} the PA effective dose.

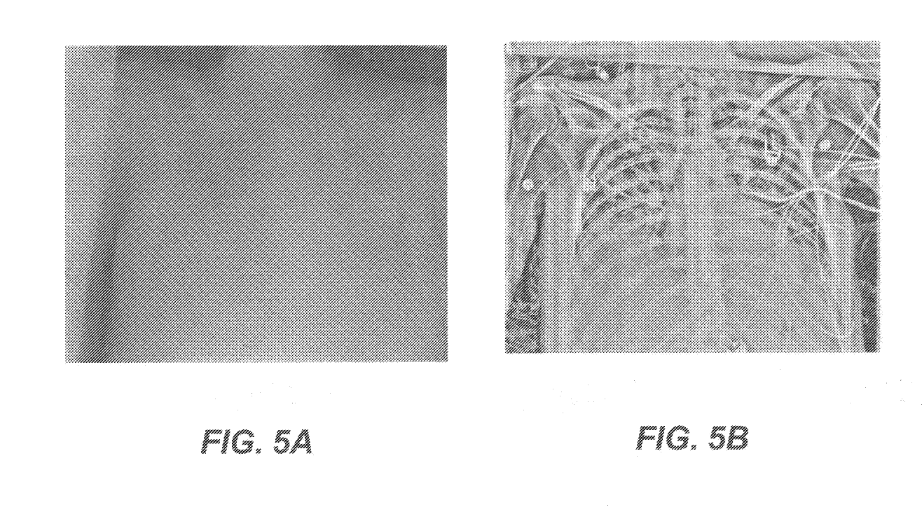

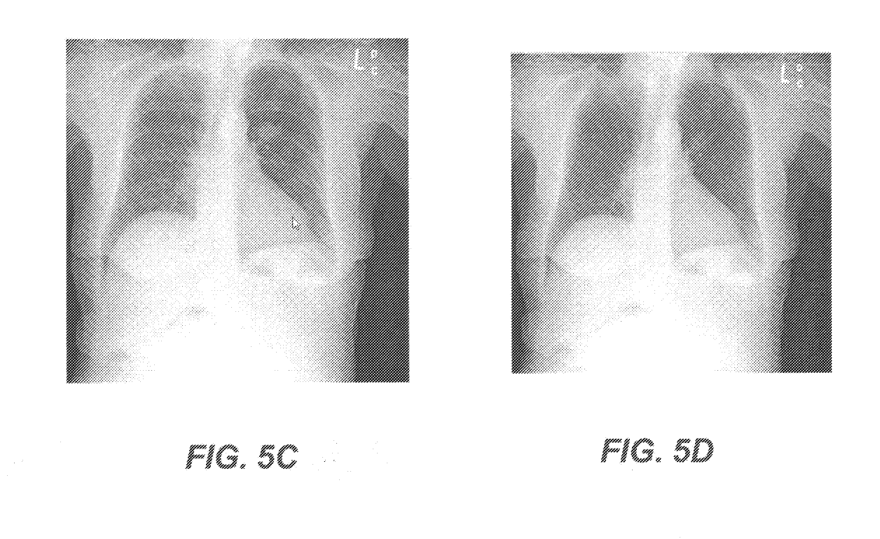

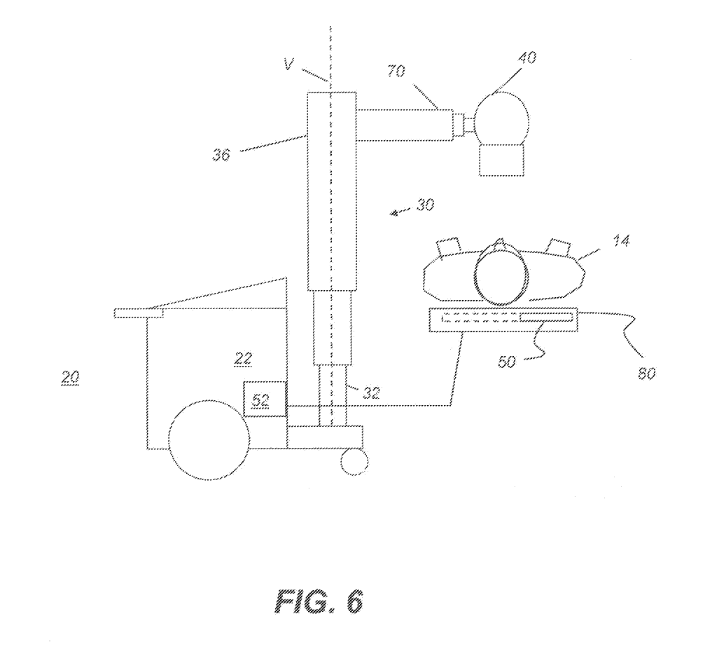

As noted previously, frequency decomposition can be an effective tool for showing RSFOs more clearly. By way of example, FIG. 5A shows a conventional fluoroscopy image, exhibiting high noise content and low contrast. FIG. 5B shows an image with different rendering for the same or similar anatomy, showing the effects of multi-band spatial frequency decomposition for enhancing visibility of tubing, wires, clips, and other features in the image content. It can be appreciated that use of multi-band spatial frequency decomposition techniques can be advantageous for enhancing the visibility and detectability of an RSFO in the patient image.

Reference is made to commonly assigned U.S. Pat. No. 7,848,560 (Wang), incorporated herein in its entirety by reference.

### DR Detector Positioning

Tomosynthesis imaging using a portable imaging apparatus can have particular value for acquiring images of the surgical ROI that allow analysis for foreign materials or objects such as RSFOs. As noted previously, tomosynthesis obtains a number of images of a region of interest, each image at a different angle, providing a measure of depth information. While not equivalent to the 3-D or volume imaging results obtained using CT or CBCT imaging, tomosynthesis imaging provides at least some amount of depth information over 2-D x-ray imaging. Volume reconstruction algorithms for tomosynthesis enable visualization of 3-D objects, but without high depth resolution.

For RSFO detection, even a reduced amount of depth information when compared to tomosynthesis can be useful, without the requirement for image reconstruction.

Tomosynthesis imaging of an ROI can be achieved by changing the relative positions of the radiation source and image detector. That is, either or both the source and detector can be moved to a different relative position for acquiring each projection image. The imaging apparatus 60 shown in FIG. 2D shows an arrangement of source array 44 in which the angle of the radiation source 48 effectively changes from one image to the next. FIG. 6 shows an alternate arrangement for tomosynthesis, in which radiation source 40 is stationary while detector 50 is shifted to different relative positions by a transport apparatus 80.

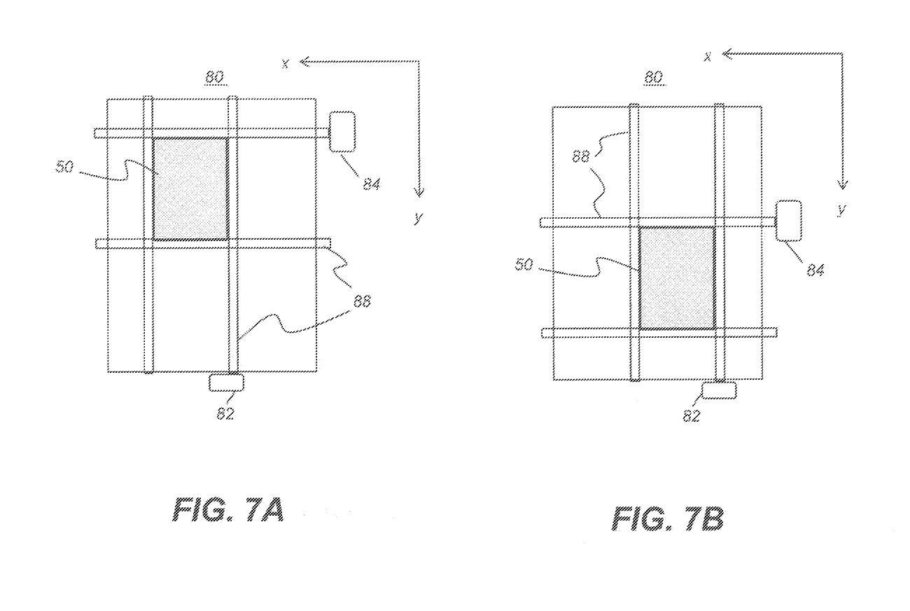

FIGS. 7A and 7B show top views of transport apparatus 80 with detector 50 moved to different positions. With respect to the orientation of apparatus 80 shown in FIG. 6, these positions are horizontal (normal to the page). In this arrangement, detector 50 is mounted on orthogonal rails 88, driven in x and y directions by one or more actuators 82, 84 under control of processor 52 (FIG. 6). Using a mechanical arrangement of this type, portable imaging apparatus 20 can obtain multiple images at different angles in order to acquire tomosynthesis projection image data that can be used for RSFO detection.

Computer-guided positioning can be provided for moving the detector 50 into an appropriate position for imaging.

### Surgical Support Workflow

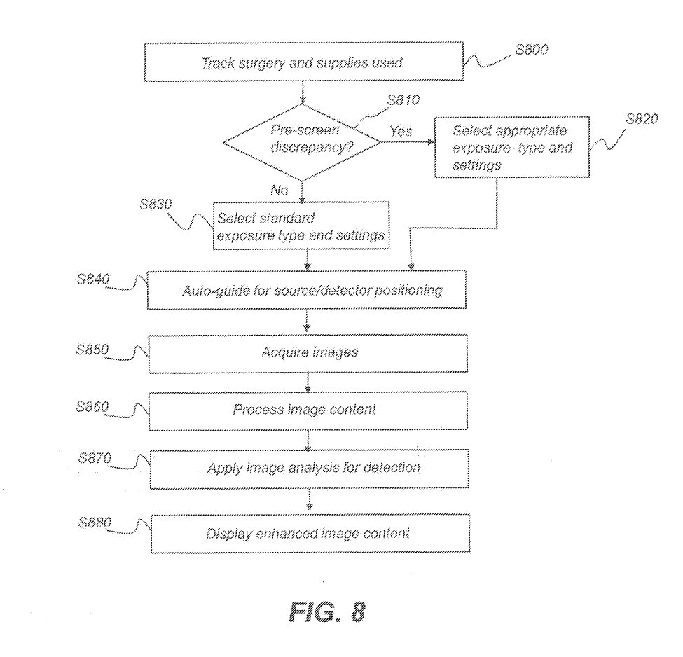

The logic flow diagram of FIG. 8 shows a sequence for RSFO detection according to an embodiment of the present disclosure. Processing for this sequence is executed by radiography system 200 (FIG. 2B) and can be assisted by one or more networked computers or dedicated logic processors that are in signal communication with radiography system 200. In an optional initial tracking step S800, the system obtains information on supplies disposition that is useful for setting subsequent operational parameters for RSFO detection. Tracking step S800 obtains information on the type of surgical procedure and on what instruments and materials are used by the surgical team. This can be information tracked using radio-frequency identification (RFID) tags detected by tracking mechanism 18 (FIG. 2B), information manually entered by a surgical team member, or information obtained from a database of a priori information about instruments typically used for particular types of surgery, for example. Optionally, one or more cameras or other reflectance imaging apparatus can be used as part of tracking mechanism 18, along with audio recording, to record use of different tools and materials during the procedure. Cameras disposed at different angles relative to the patient can be advantageous for providing triangularization data, for example. As part of tracking step S800, the surgical team can review recorded information related to surgical supplies used and their disposition following surgery. This can include verifying any accounting performed by the system, for example.

According to an embodiment of the present disclosure, RSFO tracking in step S800 can use both audio (voice) and video (image) tracking of the surgical procedure in order to detect which surgical supplies were used and to track their disposition following surgery. Thus, for example, image analysis software that supports the tracking function can detect which tools or materials are handled by the surgical team and used within and around the surgical site. One or more cameras, for example, can be provided for obtaining image content continuously or at regular intervals during surgery. Audio recording can be continuously monitored and verbal data recorded in order to support the supplies tracking function. This tracking function can provide information not only on which supplies were used, but also on their disposal following surgery.

At the conclusion of surgery, a prescreening check step S810 executes. If pre-screening indicates some possible discrepancy between routine checks performed by the surgical team and tracking information obtained in step S800, the system determines a course of image acquisition and processing activity. The strategy that is used can be based on whether there appears to be discrepancy between counts maintained for surgical instruments and devices that are dense and radio-opaque, or whether the discrepancy relates to sponges, gauze pads, and other soft materials that are not as readily detectable using x-rays. Where there is discrepant data, a type and settings selection step S820 then selects appropriate exposure type and settings depending on the nature of the discrepant information. According to an embodiment of the present disclosure, image acquisition may obtain one of the following types of images:

(i) x-ray image;

(ii) dual-energy x-ray image, which consists of two images, one at a lower exposure level, the other at higher exposure;

(iii) tomosynthesis images at two or more different angles with respect to the surgical ROI.

If data suggests that a surgical tool or instrument of some type may not be accounted for, exposure type and settings for dense, radio-opaque devices and features are automatically selected for use by the system. Thus, for a surgical instrument, a single, higher energy x-ray image may be sufficient. If, on the other hand, data suggest that a sponge or gauze pad may have been retained, alternate settings for type and exposure can be automatically set for subsequent image acquisition. Low-dose tomosynthesis or dual energy radiography may be more appropriate as an imaging type in such a case.

According to an embodiment of the present disclosure, the imaging system that is used for RSFO detection provides various types of prompt messages as a result of tracking activity. These messages can include audio messages that suggest specific locations of materials used during surgery. Audible messages can be provided to remind the surgical team of the location(s) of various instruments, sponges, gauze pads, needles, or other materials that were used, as determined by tracking image analysis or other utility, such as using an RFID detector or other tool. Visual and audio data acquired during the surgery can be correlated with other tracking mechanisms and indicia in order to provide more accurate tracking data. Voice analysis can obtain information on surgical supplies use and final disposition. Alternately, prompt messages can be displayed on-screen as text for the operator, for example.

Where no discrepancy is detected, an alternate type and settings selection step S830 executes, specifying standard exposure type and settings. These standard settings may vary for individual body dimensions or for surgery type or anatomy, for example, but can be standardized for detection of either surgical instruments or soft materials. Standard settings can be default settings for image type and exposure that are automatically used following the surgical procedure, unless otherwise replaced by settings that are optimized for detection of particular surgical components or materials.

Continuing with the sequence of FIG. 8, once settings have been automatically made for post-operative imaging, an optional auto-positioning step S840 can be executed. Some type of positioning mechanism, such as that described previously with reference to FIGS. 7A and 7B, can be used to properly position the DR detector for imaging the surgical ROI. Positioning used for the same anatomy can differ based on imaging type, such as where tomosynthesis is used, for example. Auto-positioning step S840 can also adjust a collimator for FOV adjustment. An image acquisition step S850 follows, obtaining the type of radiographic images deemed appropriate for the particular surgery procedure, anatomy region, discrepancy type, materials type, and other factors. An optional image processing step S860 processes the image content, such as to apply bone/rib suppression or other image conditioning that can assist in foreign objects detection.

A subsequent image analysis step S870 then provides the automated detection utilities that enable processing logic to detect any non-normal tissue features or other features that can indicate a candidate surgical foreign object, such as pads, sponges, supplies, or instruments, for example. Image analysis step S870 can use the CAD utilities described previously for determining features of the surgical ROI that can indicate candidate foreign objects. In addition, image analysis can also used bone/rib suppression and other utilities that help to suppress image content for particular features of the surgical ROI. A display step S880 then displays imaging and analysis results, enhancing or highlighting image content for viewing by the surgical staff.

According to an embodiment of the present disclosure, fluoroscopy imaging can also be provided by the imaging apparatus. By its nature, fluoroscopy is not optimized to provide sufficient image quality for automated image acquisition and display or for highly accurate analysis of image content, such as that useful for RSFO detection. However, fluoroscopy can be a useful utility for detection of surgical supplies under some conditions.

Embodiments of the present disclosure are not intended to replace conventional surgical practices that account for the disposition of surgical supplies following surgery. The apparatus and method of the present disclosure can supplement existing procedures, providing additional and corroborative information that can help the surgical staff to more accurately assess whether or not there is need for concern about possible RSFOs following the operation. The system identifies candidate RSFOs; it remains to the surgical team or other practitioners to determine the likelihood of an actual retained device and to identify a course of action.

According to an embodiment of the present disclosure, one or more radiographic images of the surgical ROI can be obtained in the operating room itself, without requiring movement of the patient to a separate facility. Processing hardware on the portable imaging apparatus itself, such as on one of the apparatus arrangements described with reference to FIGS. 2A-2E, for example, can then be used to perform the image analysis described with reference to FIG. 3 for identifying likely RSFOs. The image processing can be performed on the portable imaging apparatus or the image data can be processed at a networked computer that is in signal communication with the portable imaging apparatus.

### Image Processing Variations

According to an embodiment of the present disclosure, different types of image processing can be used to analyze and report results as part of the RSFO assessment process. The portable radiographic imaging apparatus may apply any of various approaches to RSFO detection, such as based on tissue texture, density, or other factors discernable from the acquired image content. Networked computer resources may alternately be available to analyze image content using other analysis strategies, including use of parts libraries that store data on surgical instruments and materials used at a facility. Other networked systems may have PACS (picture archiving and communications systems) access that enables analysis to use information previously obtained from a particular patient or from atlas or other information based on a statistical population and to use this information for comparison with newly obtained image content.

Applicants have described an imaging method, comprising: capturing at least one tomosynthesis image of a patient; and analyzing the captured at least one tomosynthesis image to detect a surgical instrument. The analysis can employ a computer-aided detection (CAD) algorithm/system or other knowledge-based or expert-based system.

Applicants have further described an imaging method, comprising: capturing at least one dual energy (DE) image of a patient; and analyzing the captured at least one DE image to detect a surgical object. The analysis can employ a computer-aided detection (CAD) algorithm/system or other knowledge-based or expert-based system.

Applicants have described an imaging method, comprising: capturing at least tomosynthesis image and at least one dual energy image of a patient; and analyzing the captured at least one tomosynthesis image and the captured at least one dual energy image to detect a surgical instrument. The analysis can employ a computer-aided detection (CAD) algorithm/system or other knowledge-based or expert-based system.

Applicants' imaging method can further comprise: displaying the at least one captured image; and indicating a location of the detected surgical instrument within the displayed image. The indication can include an arrow, symbol/marker, bolding, highlighting, coloring, outlining, or the like.

Applicants have described a method for tracking the disposition of surgical supplies in an operating room, the method executed at least in part by a computer and comprising: (i) tracking the disposition of surgical supplies used in an operation by capturing images from at least one camera and recording audio from a surgical team; (ii) analyzing the images and audio to detect a discrepancy related to surgical supplies use in the tracked operation; and (iii) prompting the surgical team with audible or visual information on one or more surgical supplies.

An embodiment of the present disclosure can be a software program. Those skilled in the art can recognize that the equivalent of such software may also be constructed in hardware. Because image manipulation algorithms and systems are well known, the present description is directed in particular to algorithms and systems forming part of, or cooperating more directly with, the method in accordance with the present invention. Other aspects of such algorithms and systems, and hardware and/or software for producing and otherwise processing the image signals involved therewith, not specifically shown or described herein may be selected from such systems, algorithms, components and elements known in the art.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

The methods described above may be described with reference to a flowchart. Describing the methods by reference to a flowchart enables one skilled in the art to develop such programs, firmware, or hardware, including such instructions to carry out the methods on suitable computers, executing the instructions from computer-readable media. Similarly, the methods performed by the service computer programs, firmware, or hardware are also composed of computer-executable instructions.

In this document, the terms “a” or “an” are used, as, is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim.

The system/method has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

The invention has been described in detail, and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. An imaging method, executed at least in part by a computer, comprising:
tracking the disposition of surgical supplies used in an operation;
identifying a radiographic imaging technique for detecting a retained surgical foreign object according to the tracking;
acquiring one or more radiographic images in the operating room;
analyzing the acquired image content to identify one or more candidate foreign objects;
displaying at least a portion of the acquired image content and highlighting the one or more candidate foreign objects in the acquired image content.

2. The method of claim 1 wherein tracking the disposition of surgical supplies comprises obtaining one or more camera images.

3. The method of claim 1 wherein tracking the disposition of surgical supplies comprises recording audio.

4. The method of claim 1 wherein identifying the radiographic imaging technique identifies one of x-ray imaging, dual-energy imaging, and tomosynthesis.

5. The method of claim 1 further comprising acquiring the one or more radiographic images in the operating room at same or different positions of a radiation source or a detector.

6. The method of claim 1 further comprising adjusting a collimator to adjust the field of view for acquiring the one or more radiographic images.

7. The method of claim 1 wherein highlighting the one or more candidate foreign objects comprises displaying an outline.

8. The method of claim 1 wherein tracking the disposition of surgical supplies comprises recording manual entry of information from an operator.

9. The method of claim 1 wherein identifying the radiographic imaging technique is performed according to results of the tracking.

10. The method of claim 1 wherein analyzing the acquired image content further comprises suppressing bone content.

11. The method of claim 1 wherein analyzing the acquired image content further comprises suppressing tissue content.

12. A portable imaging apparatus comprising:
a mobile cart having a radiography source and a control processor;
a digital radiography receiver that is in signal communication with the control processor; and
an operator interface with an operating mode for on-site imaging of a surgical region of interest, wherein the control processor is configured to execute instructions that analyze image content from the digital radiography receiver and report and display a detected retained surgical foreign object.

13. The apparatus of claim 12 wherein the operating mode is selected from a group consisting of x-ray imaging, dual-energy x-ray imaging, and tomosynthesis imaging.

14. The apparatus of claim 12 further comprising a transport for movement of the digital radiography receiver with respect to the surgical region of interest.

15. An imaging method, comprising:
capturing at least one tomosynthesis image of a patient; and
analyzing the captured at least one tomosynthesis image to detect a surgical instrument or material.

16. The imaging method of claim 15, further comprising:
capturing at least one dual energy image of a patient; and
analyzing the captured at least one tomosynthesis image and at least one dual energy image to detect the surgical instrument or material.

17. The imaging method of claim 15 wherein analyzing includes using a computer-aided detection (CAD) algorithm/system or other knowledge-based expert system.

18. The imaging method of claim 15 wherein the surgical instrument is a sponge or gauze pad.

19. The imaging method of claim 15 further comprising the step of applying bone and soft tissue suppression to the captured image prior to analyzing the captured image.

20. The imaging method of claim 15 wherein:
capturing at least one tomosynthesis image of a patient comprises:
acquiring a first tomosynthesis projection image of a surgical region of interest of the patient at a first acquisition angle; and
acquiring a second tomosynthesis projection image of the surgical region of interest of the patient at a second acquisition angle; and
wherein analyzing the captured at least one tomosynthesis image comprises:
analyzing the first and second tomosynthesis projection images to detect the surgical instrument or material in the surgical region of interest.

21. An imaging method, comprising:
capturing at least one image of a patient, the image being a tomosynthesis image or a dual energy image; and
analyzing the captured at least one image to detect a surgical instrument or material.

22. The imaging method of claim 21, further comprising:
displaying the at least one captured image; and
indicating a location of the detected surgical instrument or material within the displayed image.

23. The imaging method of claim 21, wherein the indicating includes indicating with a mark, the mark being any one of: arrow, symbol/marker, bolding, highlighting, coloring, outlining, or the like.
